(19) United States
(12) Patent Application Publication
(10) Pub. No.: US 2025/0025148 A1
(43) Pub. Date: Jan. 23, 2025

(54) SURGICAL INSTRUMENT

(71) Applicants: COUSIN BIOTECH, WERVICQ SUD (FR); HOOGLAND SPINE PRODUCTS, UNTERFÖHRING (DE)

(72) Inventors: Lucie BECQUART, LILLE (FR); Eléonore HOFFMANN, WAMBRECHIES (FR); Michel CAILLIBOTTE, LILLE (FR); Jaap HOOGLAND, MÜNCHEN (DE)

(21) Appl. No.: 18/707,091

(22) PCT Filed: Nov. 8, 2022
(86) PCT No.: PCT/EP2022/081063

§ 371 Date: May 2, 2024

(30) Foreign Application Priority Data
Nov. 10, 2021 (FR) FR2111945

(51) Int. Cl. A61B 17/02

(52) U.S. Cl. CPC A61B 17/025; A61B 17/0218; A61B 2017/0256

(57) ABSTRACT

The present invention relates to a surgical instrument (10) comprising a cannula (20) comprising a conduit (30), said conduit (30) comprising an inlet orifice (40) and an outlet orifice (50), said cannula (30) having a longitudinal axis L. Said instrument (10) further comprises first (60) and second (90) distraction rods comprising respectively first and second intermediate portions (65, 95) with longitudinal axes L1 and L2. The first and second distraction rods (60, 90) are pivotally mounted respectively about the longitudinal axes L1 and L2 and comprise respectively a first distal part (62) and a second distal part (92) configured so that the pivoting of the first and second distraction rods (60, 90) about their respective longitudinal axes L1 and L2 causes the displacement of first and second distal parts (62, 92) closer to or away from each other.

### TECHNICAL FIELD

The present invention relates to a surgical instrument making it possible to retract the soft tissues and place an implantable device on an operative site, particularly in an interlaminar or interspinous space of the spine, said instrument being configured to also allow distraction of two regions of the implantation area, particularly a distraction of two regions of the spine.

### PRIOR ART

We know instruments of the tubular retractor type that are intended to be used in spine surgery on a patient and making it possible to maintain a dilation of the soft tissues and a space allowing access to the interlaminar or interspinous surgical site for implant placement.

These tubular retractors make it possible to maintain a passage extending from outside the patient's body to a cavity of the patient's body, particularly at a particular location where the surgical procedure is to be performed. The passage for the placement of a tubular retractor into the patient's body is first created using a procedure implementing cannulated dilators with increasing diameters.

A typical procedure consists in advancing a guidewire through a small incision which is advanced to the lower edge of the upper lamina of the spine. A small incision which extends cranially or caudally is then made.

A first cannulated dilator is then inserted over the guidewire. Larger-diameter cannulated dilators are then inserted sequentially on top of each other to increase the opening into the body cavity. Typically, the guidewire is removed after installation of the first cannulated dilators and finally the cannulated dilators are all removed after a tubular retractor has been inserted onto the largest of the cannulated dilators.

Once these procedures have been completed, the tubular retractor is locked in position by a retractor forceps which is clamped on a retractor clamping arm which is fixed to a rigid element such as the operating table. This disposition provides an unhindered area where the surgeon can perform the medical procedure. However, the retraction of the soft tissues is fixed, and is determined by the diameter of the tubular retractor placed after removal of the cannulated dilators. These instruments do not allow adjusting the retraction between two regions of the area to be treated.

As is well known in this technical field, the cannulated dilators and the tubular retractors are manufactured from a surgical metal material and are dimensioned for the greatest depth provided in the surgical procedure.

For example, in current lumbar discectomy practices whose objective is to decompress the affected nerve root, endoscopic and microendoscopic techniques are often used. These techniques are becoming popular insofar as the patient benefits from a very small incision and limited tissue disruption. The surgeon benefits from better visualization and better lighting thanks to instruments of the tubular retractor type. These advantages make it possible to shorten the hospital stay and speed up the patient's recovery. A lumbar discectomy can be performed by using a tubular retractor, but it is possible to perform a laminotomy, a facetectomy, a foraminotomy, or simply a nerve root retraction.

We also know instruments of the retractor type allowing distraction of two regions of the spine, such as the one described in patent application WO 2010136860. In this document, the instrument (see [FIG. 3]) makes it possible to adjust the retraction between the spinous processes in order to clear the interlaminar space. This distraction also concomitantly causes a very significant enlargement of the surgical skin incision and the soft tissues. The distraction is therefore obtained after making a relatively large skin incision that is traumatic to the tissues. This technique, even if it is considered non-invasive, still remains more invasive than the dilatation technique implementing a final tubular retractor.

The tubular retractor type instruments however have the drawback of not making it possible to also perform an adjustable distraction between two regions of the area to be treated, particularly to adjust the distance separating two regions of the area to be treated.

There is therefore a need for a surgical instrument making it possible at the same time to retract the soft tissues from a site to be treated by limiting the size of the skin incision, and at the very least allowing it to remain fixed, to convey and place an implant on the site to be treated, while making it possible to adjust the retraction between two regions of the area to be treated.

### DISCLOSURE OF THE INVENTION

The present invention relates, according to a first aspect, to a surgical instrument overcoming all or part of the aforementioned problems, advantageously comprising:
cannula comprising a conduit, said conduit comprising an inlet orifice and an outlet orifice, said cannula having a longitudinal axis L;
a first distraction rod comprising a first intermediate portion with a longitudinal axis L1 and a first distal part, and
a second distraction rod comprising a second intermediate portion with a longitudinal axis L2 and a second distal part,
preferably said axes L1 and L2 are parallel to each other and parallel to the longgitudinal axis L of the cannula.

In addition, the first distraction rod is pivotally mounted about the longitudinal axis L1, and the second distraction rod is pivotally mounted about the longitudinal axis L2; and the first distal part and the second distal part are configured so that the pivoting of the first distraction rod about the longitudinal axis L1 and/or the pivoting of the second distraction rod about the longitudinal axis L2 cause(s) the displacement of the first and second distal parts closer to or away from each other.

Advantageously, the instrument according to the invention comprises a cannula making it possible to retract the soft tissues, particularly at least according to a distance corresponding to the external diameter of the cannula, and comprises first and second distraction rods in connection with the cannula, particularly pivotally mounted about the longitudinal axis L of the cannula, making it possible to retract two regions from the implantation area to be treated.

Advantageously, the cannula comprises a hollow conduit allowing the passage of an implant, possibly folded on itself, through the latter. Particularly, the implant is introduced into the conduit by passing it through the inlet orifice of the cannula, then moved inside the conduit, particularly pushed using a surgical tool, to the outlet conduit of the conduit, leading to the implantation area to be treated.

Advantageously, the first distraction rod and/or the second distraction rod can pivot (each) equally in the trigonometric direction and in the direction opposite to the trigonometric direction.

Advantageously, the first distraction rod and the second distraction rod pivot along their respective longitudinal axes L1 and L2 independently of each other.

Advantageously, the first distraction rod may comprise one or more longitudinal axis(es), the longitudinal axis L1 of its first intermediate portion, and possibly the longitudinal axis Lbr1 of its first branch and/or the longitudinal axis Lbr2 of its second branch.

Advantageously, the longitudinal axis L1 passes through the central axis of the first intermediate portion.

Advantageously, the second distraction rod comprises one or more longitudinal axis(es), the longitudinal axis L2 of its second intermediate portion, and possibly the longitudinal axis Lbr3 of its first branch and/or the longitudinal axis Lbr4 of its second branch.

Advantageously, the longitudinal axis L2 passes through the central axis of the second intermediate portion.

Advantageously, the longitudinal axis L1 is the rotational or pivotal axis of the first intermediate portion.

Advantageously, the longitudinal axis L2 is the rotational or pivotal axis of the second intermediate portion.

Advantageously, it is understood by the first distraction rod pivotally mounted along the axis L1 that the first distraction rod is mounted so as to rotate about the axis L1.

Advantageously, it is understood by the second distraction rod pivotally mounted along the axis L2 that the second distraction rod is mounted so as to rotate about the axis L2.

Advantageously, when the first and second distraction rods pivot in opposite directions of rotation so as to make the first and second distal parts converge, the first and second distal parts move closer to each other, particularly they are in contact at least partly with each other or are contiguous. This position of the first and second distal parts advantageously corresponds to an initial position, also designated in the present text by position B or position “0”. In this position, since the first and second distal parts are in contact with each other, it is known that the spacing between them is zero. This is a calibration position, so that any distraction movement of the first and second parts from this initial position makes it possible to accurately evaluate their spacing.

Advantageously, when the first and second distraction rods pivot in opposite directions of rotation so as to make the first and second distal parts converge, the first and second distal parts move away from each other. This is a distraction position in which the first and second distal parts gradually bear on the surrounding areas of the implantation area, for example an interlaminar or interspinous bearing, and the progression of the pivoting of the first and second distraction rods makes it possible to retract at least two surrounding areas from each other, particularly the first distal part is in contact and pushes a first surrounding area and the second distal part is in contact and pushes a second surrounding area.

Advantageously, the first intermediate portion is substantially rectilinear or straight.

Advantageously, the second intermediate portion is substantially rectilinear or straight.

In one embodiment, the first intermediate portion has a length greater than or equal to 20 mm or 30 mm and less than or equal to 300 mm or 200 mm or 150 mm.

In one embodiment, the second intermediate portion has a length greater than or equal to 20 mm or 30 mm and less than or equal to 300 mm or 200 mm or 150 mm.

Preferably, the cannula comprises an outer surface and an inner surface leading to the interior volume of the conduit.

Preferably, the cannula has a preferably substantially constant or variable thickness extending between its outer surface and its inner surface.

Advantageously, the first and second intermediate portions are pivotally mounted and in connection with the external surface of the cannula.

Preferably, the cannula has an external diameter, in particular maximum diameter Dc, particularly Dc is greater than or equal to 5 mm and less than or equal to 50 mm, more particularly less than or equal to 35 mm.

In one embodiment, the cannula or the conduit has a length greater than or equal to 20 mm or 30 mm and less than or equal to 300 mm or 200 mm or 150 mm.

In one embodiment, the cannula is substantially rectilinear or straight.

Advantageously, the first and second intermediate portions are separate by a distance dsep which can be greater than or equal to Dc, or less than or equal to twice Dc, or even less than or equal to DC.

This spacing depends on the extent of the desired distraction.

In one embodiment, dsep is less than or equal to Dc in order to reduce the space requirement of the instrument.

Preferably, the distance, in particular the maximum distance, separating the first and second distal parts, particularly the first and second free ends respectively of the first and second distal parts, is comprised between 0 mm and 100 mm, preferably between 0 mm and 75 mm, still preferably between 0 mm and 60 mm.

Preferably, the first distraction rod has the shape of a substantially cylindrical, in particular solid, rod.

Preferably, the second distraction rod has the shape of a substantially cylindrical, in particular solid, rod.

Preferably, the first distraction rod has an external diameter greater than or equal to d1tg, particularly greater than or equal to 2 mm, more particularly less than or equal to 8 mm.

Preferably, the second distraction rod has an external diameter greater than or equal to d2tg, particularly greater than or equal to 2 mm, more particularly less than or equal to 8 mm.

Preferably, the cannula and the first and second distraction rods are in one or more material(s) that can be used as implantable material(s) or as a surgical instrument, for example in stainless steel and/or one or more medical grade polymer material(s).

In one variant of embodiment, the first distal part comprises a first branch having a longitudinal axis Lbr1 secant with the longitudinal axis L1 of the first intermediate portion.

Advantageously, the first branch of the first distal part has a longitudinal axis Lbr1 forming an angle α1 with the longitudinal axis L1 of the first intermediate portion, preferably the angle α1 is greater than or equal to 30°,particularly less or equal to 120°, more particularly less than or equal to 90° or 70°.

Advantageously, the first distal part comprises a first curvature having an angle α1 greater than or equal to 30°, particularly less than or equal to 120°, more particularly less than or equal to 90° or 70°.

Advantageously, the first branch of the first distal part is substantially rectilinear or straight.

In one embodiment, the first branch of the first distal part has a length greater than or equal to 5 mm or 10 mm or 20 mm or less than or equal to 80 mm or 70 mm or 60 mm or 40 mm or 30 mm.

Advantageously, the first distraction rod comprises a first intermediate portion, substantially rectilinear or straight, and a first distal part in the extension of the first intermediate portion, and comprising a first curvature, and possibly a second curvature as described below.

Advantageously, the first branch of the first distal part projects outside the outlet orifice of the cannula.

In one embodiment, the second distal part comprises a first branch with a longitudinal axis Lbr3 secant with the longitudinal axis L2 of the second intermediate portion.

Advantageously, the first branch of the second distal part has a longitudinal axis Lbr3 forming an angle α3 with the longitudinal axis L2 of the second intermediate portion, preferably the angle α3 is greater than or equal to 30°, particularly less or equal to 120°, more particularly less than or equal to 90° or 70°.

Advantageously, the second distal part comprises a first curvature having an angle α3 greater than or equal to 30°, particularly less than or equal to 120°, more particularly less than or equal to 90° or 70°.

Advantageously, the first branch of the second distal part is substantially rectilinear or straight.

In one embodiment, the first branch of the second distal part has a length greater than or equal to 5 mm or 10 mm or 20 mm or less than or equal to 80 mm or 70 mm or 60 mm or 40 mm or 30 mm.

Advantageously, the second distraction rod comprises a second intermediate portion, substantially rectilinear or straight, and a second distal part in the extension of the second intermediate portion and comprising a first curvature, and possibly a second curvature as described below.

Advantageously, the first branch of the second distal part projects outside the outlet orifice of the cannula.

In one variant of embodiment, the first distal part comprises a second branch having a longitudinal axis Lbr2 secant with the longitudinal axis Lbr1 of the first branch.

Advantageously, the second branch of the first distal part has a longitudinal axis Lbr2 forming an angle β1 with the longitudinal axis Lbr1 of the first intermediate portion.

Advantageously, the longitudinal axis Lbr2 of the second branch of the first distal part is substantially parallel to the axis L1 of the first intermediate portion of the first distraction rod.

Advantageously, the first distal part comprises a second curvature having an angle β1 greater than or equal to 30°, particularly less than or equal to 120°, more particularly less than or equal to 90° or 70°.

Advantageously, the second branch of the first distal part is substantially rectilinear or straight.

Preferably, the second distal part comprises a second branch having a longitudinal axis Lbr4 secant with the longitudinal axis Lbr3 of the first branch.

Advantageously, the second branch of the second distal part has a longitudinal axis Lbr4 forming an angle β2 with the longitudinal axis L2 of the second intermediate portion.

Advantageously, the longitudinal axis Lbr4 of the second branch of the second distal part is substantially parallel to the axis L2 of the second intermediate portion of the second distraction rod.

Advantageously, the second distal part comprises a second curvature having an angle β2 greater than or equal to 30°, particularly less than or equal to 120°, more particularly less than or equal to 90° or 70°.

Advantageously, the second branch of the second distal part is substantially rectilinear or straight.

Advantageously, the first distal part of the first distraction rod comprises a first free distal end, and:
the second curvature of the first distal part is disposed between the first free distal end and the first intermediate portion of the first distraction rod, and/or
the second curvature of the first distal part is disposed between the first free distal end and the first curvature of the first distal part.

Advantageously, the second distal part of the second distraction rod comprises a second free distal end, and:
the second curvature of the second distal part is disposed between the second free distal end and the second intermediate portion of the second distraction rod, and/or
the second curvature of the second distal part is disposed between the second free distal end and the first curvature of the first distal part.

In one variant of embodiment, the first distal part comprises a first free distal end corresponding to a free end of the second branch of said first distal part.

Advantageously, the second distal part comprises a second free distal end corresponding to a free end of the second branch of the second distal part.

In one variant of embodiment, the first distal part has a substantially L shape.

Advantageously, the second distal part has a substantially L shape.

In one variant of embodiment, the first intermediate portion of the first distraction rod is mounted by bearing on the cannula.

Preferably, the first intermediate portion is mounted by bearing on the external surface of the cannula.

Advantageously, the second intermediate portion of the second distraction rod is mounted by bearing on the cannula.

Preferably, the second intermediate portion is mounted by bearing on the external surface of the cannula.

Advantageously, the cannula serves as a support for the first and second intermediate portions, particularly for the first and second sheaths receiving them.

In one variant of embodiment, the surgical instrument comprises a first sheath secured to the cannula and comprising an interior volume receiving at least partly the first intermediate portion of the first distraction rod.

Advantageously, the first intermediate portion pivots on itself along the longitudinal axis L1 inside the first sheath.

Preferably, the first sheath is secured to the cannula, particularly to the external surface of the cannula, in particular by welding or by any other technique known to those skilled in the art.

Advantageously, the first sheath has the shape of a substantially hollow cylinder.

Preferably, the first sheath has a length greater than or equal to 20 mm or 30 mm and less than or equal to 300 mm or 200 mm or 150 mm.

Advantageously, the surgical instrument comprises a second sheath secured to the cannula and comprising an interior volume receiving at least partly the second intermediate portion of the first distraction rod.

Advantageously, the second intermediate portion pivots on itself along the longitudinal axis L1 inside the first sheath.

Preferably, the second sheath is secured to the cannula, particularly to the external surface of the cannula, in particular by welding or by any other technique known to those skilled in the art.

Advantageously, the second sheath has the shape of a substantially hollow cylinder.

Preferably, the second sheath has a length greater than or equal to 20 mm or 30 mm and less than or equal to 300 mm or 200 mm or 150 mm.

In one variant of embodiment, the first distal part projects outside said first sheath.

Advantageously, the second distal part projects outside the second sheath.

In one variant of embodiment, the cannula comprises a distal cannula part cut substantially at an angle, said distal cannula part comprising a first lateral cannula part with a profile similar to the profile of the first distal part of the first distraction rod.

Advantageously, said cannula part comprises a second lateral cannula part having a profile similar to the profile of the second distal part of the second distraction rod.

Advantageously, the first lateral cannula part and the second lateral cannula part—of the distal part of the cannula—are connected by an upper edge of the outlet orifice of the cannula, and/or by a lower edge of the outlet orifice of the cannula.

Advantageously, the outlet orifice comprises a lower edge, in particular the one defined above, which is substantially planar, particularly having the shape of a spatula.

In one variant of embodiment, the surgical instrument comprises a position A in which the first distal part of the first distraction rod rests substantially by bearing on the first lateral cannula part.

Advantageously, the first distal part has a profile similar to that of the first lateral part of the cannula, so that it substantially matches the latter, such as a stowage or neutral position.

Advantageously, the surgical instrument comprises a position A in which the second distal part of the second distraction rod rests substantially by bearing on the second lateral part of the cannula.

Advantageously, the second distal part of the second distraction rod has a profile similar to that of the second lateral part of the cannula, so that it substantially matches the latter, such as a stowage or neutral position.

Indeed, in this case, the first distal part and possibly the second distal part do not obstruct the outlet orifice of the cannula, and do not project outside the space requirement of the cannula.

Advantageously, the distance between the second branch of the first distal part and the second branch of the second distal part, in the position A, is approximately on the order of the diameter of the cannula.

In one variant of embodiment, the surgical instrument comprises a position B in which the first and second distal parts are partly contiguous.

Advantageously, in the position B, the second branches of the first and second distal parts are substantially contiguous.

Advantageously, in the position B, the first and second free distal ends are contiguous.

In one variant of embodiment, the cannula has an external diameter Dc and the distance dsep separating the first and second intermediate portions of the first and second distraction rods is less than or equal to twice Dc, particularly less than or equal to Dc.

The function of retraction of the soft tissues provided by the cannula and the function of distraction of two regions of the implantation area are provided in a reduced space requirement.

Advantageously, the distraction function is provided by the first and second rods disposed on the periphery of the external surface of the cannula.

In one variant of embodiment, the first and second distraction rods comprise respectively first and second proximal parts for gripping and pivoting said first and second rods along their respective longitudinal axes L1 and L2.

Advantageously, the cannula comprises a distal part and a proximal part between which the conduit of the latter extends.

Advantageously, the first and second intermediate portions of the first and second distraction rods extend between the distal and proximal parts of the cannula.

Advantageously, the first and second distal parts of the first and second distraction rods project from the distal part of the cannula.

Advantageously, the first and second proximal parts of the first and second distraction rods project from the proximal part of the cannula.

Advantageously, the pivoting of the first and second distraction rods, and therefore the displacement of the first and second distal parts of said rods closer to or away from each other, is manually actuated by the pivoting of the first and second proximal gripping and pivoting parts.

In one variant of embodiment, the surgical instrument comprises a support, particularly a flange, through which the cannula is mounted.

Advantageously, the support comprises a distal face and a proximal face, and a through opening extending between the distal and proximal faces and leading to them.

Advantageously, the proximal part of the cannula is mounted through the through opening of the support.

Advantageously, the inlet orifice of the cannula is the through opening leading to the proximal face of the support.

Advantageously, the support comprises a first auxiliary through opening, particularly extending between the proximal and distal faces of the support and leading to them.

Advantageously, the first distraction rod comprises a portion of its length mounted through the first auxiliary opening.

Advantageously, the first proximal gripping part projects from the first auxiliary opening, particularly from the proximal face of the support.

Advantageously, the support comprises a second auxiliary through opening, particularly extending between the proximal and distal faces of the support and leading to them.

Advantageously, the second distraction rod comprises a portion of its length mounted through the second auxiliary opening.

Advantageously, the second proximal gripping part projects from the second auxiliary opening, particularly from the proximal face of the support.

In one variant of embodiment, the surgical instrument comprises a device for measuring the distance separating the first and second distal parts, in particular the distance separating the first and second free ends of the first and second distal parts, particularly the measuring device comprises a support or a flange comprising graduations.

Advantageously, said support comprises graduations arranged on its proximal face.

Advantageously, said graduations are configured to measure the spacing between the first and second distal parts, in particular between the second branches of the first and second distal parts.

In one variant of embodiment, the first and second proximal parts have a substantially bent shape.

In one variant of embodiment, the support comprises a first graduation corresponding to the position of the first distraction rod, particularly comprises a graduation 0 corresponding to the position B according to the invention.

Advantageously, the support comprises a second graduation corresponding to the position of the second distraction rod, particularly comprises a graduation 0 corresponding to the position B according to the invention.

In one variant of embodiment, the measuring device is mounted around the cannula, and said measuring device is disposed between the first intermediate portion and the first proximal part of the first distraction rod, the first distraction rod comprising a portion of its length mounted through said measuring device.

Advantageously, said measuring device is also disposed between the second intermediate portion and the second proximal part of the second distraction rod, the second distraction rod comprising a portion of its length mounted through said device.

In one variant of embodiment, the measuring device comprises a member for securing the first and second proximal ends together in a determined pivoting position, particularly comprising a rack member.

In one variant of embodiment, the surgical instrument comprises a securing area configured to cooperate with a securing tool in order to secure said instrument to a support, for example a surgical table.

### DESCRIPTION OF THE DRAWINGS

The present invention will be better understood upon reading one exemplary embodiment, cited without limitation, and illustrated by the following figures in which:

FIG. 1 schematically represents a perspective top view of one example of a surgical instrument according to the invention;

FIG. 2 schematically represents a perspective top view of the cannula and of the first and second distraction rods of the surgical instrument represented in [FIG. 1] in the position A;

FIG. 3 schematically represents a perspective top view of the cannula and of the first and second distraction rods of the surgical instrument represented in [FIG. 1] in the position B or “0”;

FIG. 4 schematically represents a perspective top view of the cannula and of the first and second distraction rods of the surgical instrument represented in [FIG. 1] in a given distraction position;

FIG. 5 schematically represents a perspective top view of the surgical instrument represented in [FIG. 1] seen along the proximal face of the support, particularly of the measuring device, on which the cannula is mounted;

FIG. 6 schematically represents a perspective view of the surgical instrument represented in [FIG. 1] positioned on the spine;

FIG. 7 schematically represents a perspective view of the surgical instrument represented in [FIG. 1] part of which positioned on the spine is more accurately represented than in [FIG. 6].

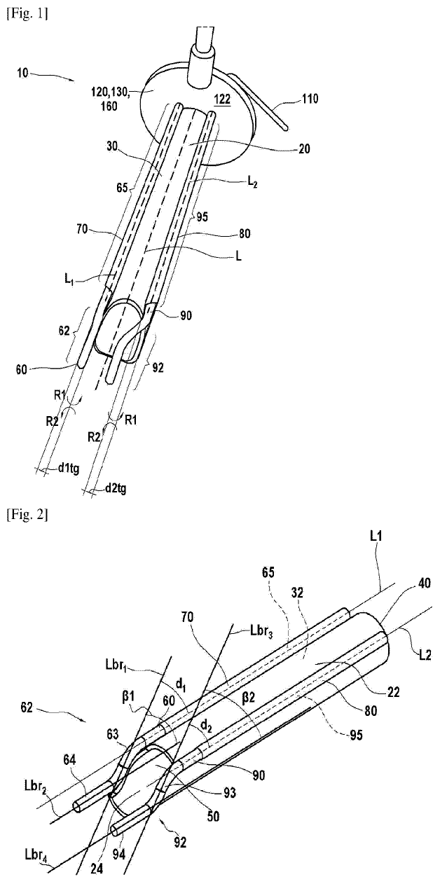

### DESCRIPTION OF THE EMBODIMENTS

The first example of surgical instrument 10 according to the invention comprises a cannula 20 comprising a conduit 30 with an inlet 40 and an outlet 50 orifice, said cannula 20 having a longitudinal axis L. The instrument 10 comprises first and second distraction rods 60, 90, which rods 60, 90 respectively comprise first and second intermediate portions 65, 95 with longitudinal axes L1 and L2, preferably parallel to each other and parallel to the longitudinal axis L of the cannula 20.

Advantageously, the first and second distraction rods 60, 90 are pivotally mounted about their respective longitudinal axes L1 and L2, and respectively comprise a first distal part 62 and a second distal part 92 configured so that the pivoting of the first and second rods 60, 90 about the longitudinal axes L1 and L2 along the directions of rotation R1 and/or R2 causes the displacement of the first and second distal parts closer to or away from each other.

Preferably, the cannula 20 comprises an outer surface 22 and an inner surface 24 leading to the interior volume 32 of the conduit 30. Preferably, the cannula 20 has a preferably substantially constant or variable thickness ec extending between its outer surface 22 and its inner surface 24. Advantageously, the first and second intermediate portions 65, 95 are pivotally mounted and in connection with the outer surface 22 of the cannula 20.

Preferably, the cannula 20 has an external in particular maximum diameter Dc, particularly Dc is greater than or equal to 5 mm and less than or equal to 50 mm, more particularly less than or equal to 35 mm. Advantageously, the first and second intermediate portions 65, 95 are separate by a distance dsep less than or equal to Dc.

Preferably, the first distraction rod 60 has an external diameter greater than or equal to d1tg, particularly greater than or equal to 2 mm, more particularly less than or equal to 8 mm.

Preferably, the second distraction rod 90 has an external diameter greater than or equal to d2tg, particularly greater than or equal to 2 mm, more particularly less than or equal to 8 mm.

Advantageously, the first and second intermediate portions 65, 95 are substantially rectilinear, the first distal part 62 is in the extension of the first intermediate portion 65 and the second distal part 92 is in the extension of the second intermediate portion 95.

Advantageously, the first distal part 62 comprises a first branch 63 having a longitudinal axis Lbr1 secant with the longitudinal axis L1. The angle formed at the intersection of the axes Lbr1 and L1 is the angle α1, preferably the angle α1 is comprised between 60° and 30°, of the order of 60° or 45° for example.

Advantageously, the angle α1 corresponds to a first curvature. Advantageously, the first branch 63 projects outside the outlet orifice 50 of the cannula 20.

The first distal part 62 comprises a second branch 64 having a longitudinal axis Lbr2 secant with the longitudinal axis Lbr1 of the first branch 63. Advantageously, the angle formed at the intersection of the axes Lbr2 and Lbr1 is the angle β1.

Advantageously, the longitudinal axis Lbr2 of the second branch 64 of the first distal part 62 is substantially parallel to the axis L1, The angle β1 corresponds to a second curvature of the first distal part 62. Preferably, the angle β1 is comprised between 60° and 30°, of the order of 60° or 45° for example.

The second distal part 92 is constructed similarly to the first distal part 62, and comprises a first branch 93 and a second branch 94 with respective longitudinal axes Lbr3 and Lbr4. The axes Lbr3 and Lbr4 respectively form angles α2 and β2 corresponding to first and second curvatures of the second distal part 92. The first and second branches 93, 94 project outside the outlet orifice of the cannula.

Advantageously, the first and second distal parts 62, 92 have L-shaped profiles. This particular shape allows the first and second distal parts 62, 92 to move closer to or away from each other while they are in the extension of the first and second intermediate portions 65, 95 which are substantially parallel.

Advantageously, the longitudinal axis Lbr4 is substantially parallel to the axis L2.

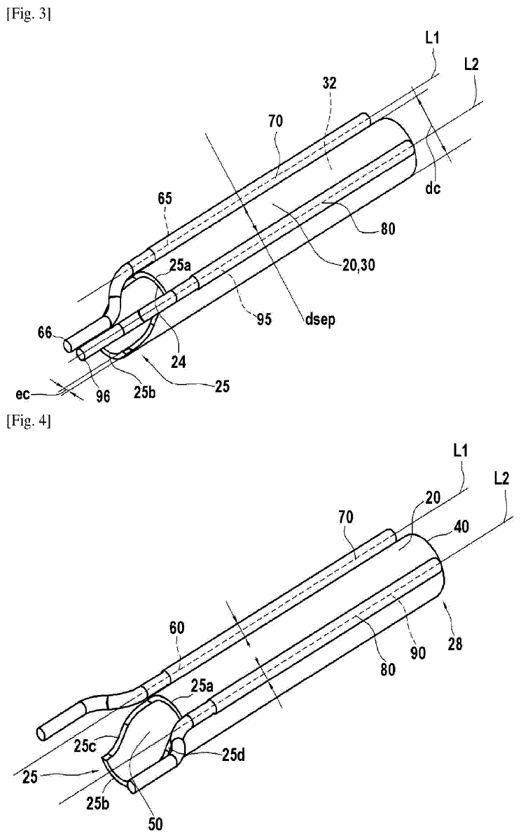

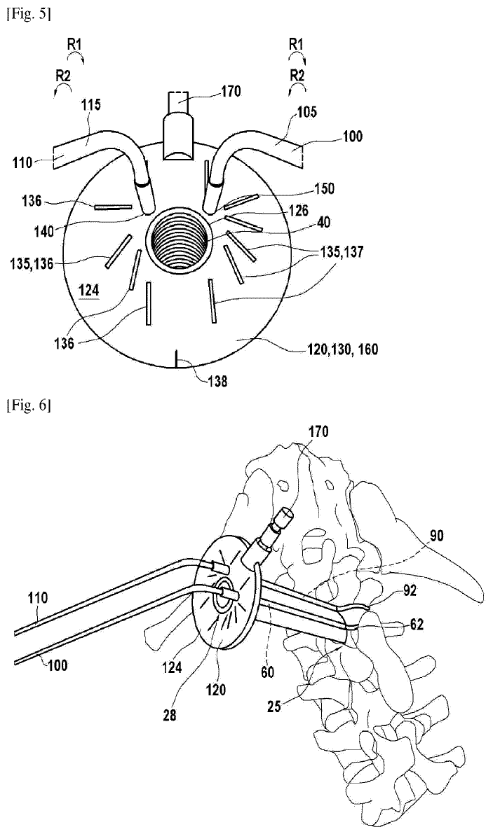

Advantageously, the first distal part 62 and the second distal part 92 of the first and second distraction rods 60, 90 comprise a first free distal end 66 and a second free end 96 (see in [FIG. 3]), particularly said free distal ends 66 and 96 are respectively the free ends of the second branches 64, 94.

Advantageously, the first and second branches 63, 64 of the first rod 60 are disposed between the distal end 66 and the first intermediate portion 65. The first and second branches 93, 94 of the second rod 90 are disposed between the distal end 96 and the second intermediate portion 95.

Advantageously, the first intermediate portion 65 is pivotally mounted inside a first sheath 70 secured to the cannula 20 and comprising an interior volume receiving at least partly the first intermediate portion 65 of the first distraction rod 60.

Advantageously, the first intermediate portion 65 pivots on itself along the longitudinal axis L1 inside the first sheath 70.

Advantageously, the second intermediate portion 95 is pivotally mounted in a second sheath 80 secured to the cannula 20 and comprising an interior volume receiving at least partly the second intermediate portion 95 of the second distraction rod 90.

Advantageously, the second intermediate portion 95 pivots on itself along the longitudinal axis L1 inside the first sheath 80.

Preferably, the first and second sheaths 70, 80 are secured to the external surface 22 of the cannula 20, in particular by welding or by any other equivalent technique known to those skilled in the art.

The first and second distal parts 62, 92 project outside their respective sheaths 70, 80.

Advantageously, the cannula 20 comprises a distal part 25 of a cannula 20 cut substantially at an angle, said distal cannula part 25 comprising a first lateral cannula part 25c, in particular a first lateral edge of the distal part 25 of the cannula 20, having a profile similar to the profile of the first distal part 62 of the first distraction rod 60.

Advantageously, said distal part 25 of the cannula 20 comprises a second lateral cannula part 25d, in particular a first lateral edge of the distal part 25 of the cannula 20, having a profile similar to the profile of the second distal part 92 of the second distraction rod 90.

Advantageously, the first lateral cannula part 25c and the second lateral cannula part 25d are connected by an upper edge 25a of the outlet orifice 50 of the cannula 20, and by a lower edge 25b of the outlet orifice 50 of the cannula 20.

Advantageously, the lower edge 25b is substantially flat, particularly has the shape of a spatula.

Advantageously, the first and second distraction rods 60, 90 respectively comprise first and second proximal parts 100, 110 for gripping and pivoting said first and second rods 60, 90 about the longitudinal axes L1 and L2.

Advantageously, the cannula 20 comprises a distal part 25 and a proximal part 28 between which the conduit 30 extends.

Advantageously, the first and second intermediate portions 65, 95 extend between the distal 25 and proximal 28 parts of the cannula 20.

Advantageously, the first and second distal parts 62, 92 of the first and second distraction rods 60, 90 project partly from the distal part 25 of the cannula 20.

Advantageously, the first and second proximal parts 100, 110 of the first and second distraction rods 60, 90 project from the proximal part 28 of the cannula 20.

Advantageously, the surgical instrument 10 comprises a support 120, particularly a flange 130, through which the cannula 20 is mounted.

Advantageously, the support 120 comprises a distal face 122 and a proximal face 124, and a through opening 126 extending between the distal 122 and proximal 124 faces and leading to them.

Advantageously, the proximal part 28 of the cannula 20 is mounted through the through opening 126 of the support 120.

Advantageously, the inlet orifice 40 of the cannula 20 is the access to the through opening 126 leading to the proximal face 124 of the support 120. The through opening 126 is therefore coaxial with the inlet orifice 40 and leads to the interior volume 32 of the conduit 30.

Advantageously, the support 120 further comprises a first auxiliary through opening 140 and a second auxiliary through opening 150, particularly both extending between the proximal 124 and distal 122 faces of the support 120 and leading to them.

Advantageously, the first distraction rod 60 and the second distraction rod 90 each comprise a portion of their length mounted through respectively the first auxiliary through opening 140 and the second auxiliary through opening 150.

Advantageously, the first and second proximal gripping parts 100, 110 project respectively from the first and second auxiliary through openings 140, 150, particularly from the proximal face 124 of the support.

Advantageously, the surgical instrument 10 comprises a device 160 for measuring the distance separating the first and second distal parts 62, 92, particularly the flange 130 comprising graduations 135, in particular arranged on the proximal face 124 of said support 120 or flange 130.

Advantageously, said graduations 135 are configured to measure the spacing between the first and second distal parts 62, 92, in particular between the second branches 64, 94 of the first and second distal parts 62, 92.

Advantageously, the first and second proximal parts 100, 110 have a substantially bent shape, facilitating their gripping and the placement of the bent parts in relation to the graduations 135.

Advantageously, the support 120 comprises a first graduation 136 corresponding to the position of the first distraction rod 60, particularly comprises a graduation 138 corresponding to the position B according to the invention described below. Advantageously, the support 120 comprises a second graduation 137 corresponding to the position of the second distraction rod 90, particularly comprises a graduation 138 corresponding to the position B described below.

Advantageously, the measuring device 160 is mounted around the cannula 20, and is disposed between the first and second intermediate portions 65, 95 and the first and second proximal parts 100, 110 of the first and second distraction rods 60, 90. The first and second distraction rods 60, 90 each comprise a portion of their length mounted through said device 160 supported by the support 120.

The support 120 comprises a member (not represented) for securing the first and second proximal ends 100, 110 together in a determined pivoting position, particularly comprising a rack member.

Advantageously, the surgical instrument 10 comprises a securing area 170 configured to cooperate with a securing tool in order to secure said instrument 10 to a fixed support, for example a surgical table.

As represented in [FIG. 2], the surgical instrument 10 comprises a position A in which the first and second distal parts 62, 92 rest substantially by bearing respectively on the first and second lateral parts 25c, 25d of the distal part 25 of the cannula 20.

Advantageously, the first and second distal parts 62, 92 have profiles similar respectively to those of the first and second lateral parts 25c, 25d of the cannula 20, so that they substantially match the lateral parts 25c, 25d, such as a stowage or neutral position.

Advantageously, in this case, the first and second distal parts 62, 92 do not obstruct the outlet orifice 50 of the cannula 20, and do not project outside the volume occupied by the cannula 20.

Advantageously, the distance between the second branches 64, 94 is substantially of the order of the diameter Dc of the cannula 20.

Advantageously, the surgical instrument 10 also comprises a position B, represented in [FIG. 3], in which the first and second distal parts 62, 92 are partly contiguous, particularly the second branches 64, 94 of the first and second distal parts 62, 92 are substantially contiguous as well as the first and second free distal ends 66.96.

In operation, a passage is created between the implantation area to be treated and the outside of the patient's body, particularly using a set of cannulated dilators of progressive diameters. The cannula 20 is thus disposed through this passage, and the dilators are removed.

Advantageously, the cannula 20 makes it possible to keep the soft tissues retracted, particularly at least according to a distance corresponding to the external diameter Dc of the cannula 20. It is then possible to release an implant on the implantation area by passing it through the inlet orifice 40 of the cannula, through the conduit 30, the latter exiting the conduit 30 through the outlet orifice 50 of the cannula 20.

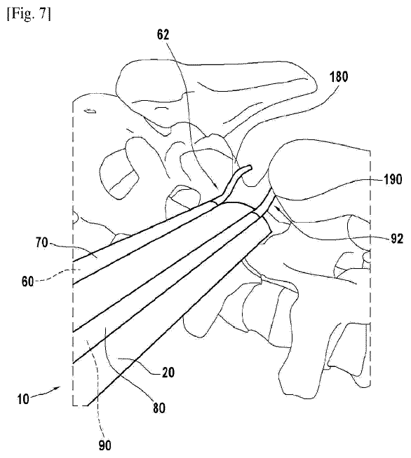

Advantageously, the instrument 10 makes it possible to retract two regions 180, 190 from the implantation area represented in [FIG. 7], in particular on the spine, for example two regions of an interlaminar or interspinous area through the first and second distraction rods 60, 90.

Preferably, the instrument 10 is inserted into the channel arranged by the cannulated distractors in position A corresponding to a neutral position and at a small space requirement of the first and second distal parts 62, 92.

Then, the operator places the instrument 10 in the position B or “0” using the first and second proximal parts 100, 110 by positioning the bent parts 105 and 115 represented in [FIG. 5] on their respective graduations (136, 137) corresponding to “0”. For this purpose, the operator pivots the proximal parts 100, 110 in opposite but converging directions (R2, R1) towards the lower central graduation 138. In this position, the operator knows that the second branches 64, 94 are substantially contiguous and are represented in [FIG. 3].

Then, the operator can begin the distraction and therefore the spacing of the first and second distal parts 62, 92 by pivoting the proximal parts 100, 110 in opposite directions according to their respective graduations 136, 137. The operator thus knows the distance separating the second branches 64, 94 thanks to the measuring device 160.

Advantageously, the function of retraction of the soft tissues provided by the cannula 20 and the function of distraction of two regions of the implantation area are provided in a reduced space requirement. The distraction function is provided by the first and second rods 60, 90 disposed on the periphery of the external surface 22 of the cannula 20.

Advantageously, the pivoting of the first and second distraction rods 60, 90, and therefore the displacement of the first and second distal parts 62, 92 of said rods 60, 90 closer to or away from each other is manually actuated by the pivoting of the first and second proximal gripping and pivoting parts 100, 110. The operator is thus guided between the positions A, B and the different distraction positions identified with the measuring device 160.

1-17. (canceled)

18. A surgical instrument wherein said surgical instrument comprises:
a cannula comprising a conduit, said conduit comprising an inlet orifice and an outlet orifice, said cannula having a longitudinal axis L;
a first distraction rod comprising a first intermediate portion with a longitudinal axis L1 and a first distal part,
a second distraction rod comprising a second intermediate portion with a longitudinal axis L2 and a second distal part,
wherein the first distraction rod is pivotally mounted about the longitudinal axis L1, and the second distraction rod is pivotally mounted about the longitudinal axis L2;
and wherein the first distal part and the second distal part are configured so that the pivoting of the first distraction rod about the longitudinal axis L1 and/or the pivoting of the second distraction rod about the longitudinal axis L2 cause(s) the displacement of the first and second distal parts closer to or away from each other.

19. The surgical instrument according to claim 18, wherein the first distal part comprises a first branch having a longitudinal axis Lbr1 secant with the longitudinal axis L1 of the first intermediate portion.

20. The surgical instrument according to claim 19, wherein the first distal part comprises a second branch having a longitudinal axis Lbr2 secant with the longitudinal axis Lbr1 of the first branch.

21. The surgical instrument according to claim 20, wherein the first distal part comprises a first free distal end corresponding to a free end of the second branch.

22. The surgical instrument according to claim 18, wherein the first distal part has a substantially L shape.

23. The surgical instrument according to claim 18, wherein the first intermediate portion of the first distraction rod is mounted by bearing on the cannula.

24. The surgical instrument according to claim 18, wherein it comprises a first sheath secured to the cannula and comprising an interior volume receiving at least the first intermediate portion of the first distraction rod.

25. The surgical instrument according to claim 24, wherein the first distal part projects outside said first sheath.

26. The surgical instrument according to claim 18, wherein the cannula comprises a distal part of the cannula cut substantially at an angle, said distal part of the cannula comprising a first lateral cannula part with a profile similar to the profile of the first distal part of the first distraction rod.

27. The surgical instrument according to claim 26, wherein it comprises a position A in which the first distal part of the first distraction rod rests substantially by bearing on the first lateral cannula part.

28. The surgical instrument according to claim 18, wherein it comprises a position B in which the first and second distal parts are partly contiguous.

29. The surgical instrument according to claim 18, wherein the cannula has an external diameter Dc and wherein the distance dsep separating the first and second intermediate portions of the first and second distraction rods is less than or equal to twice Dc.

30. The surgical instrument according to claim 18, wherein the first and second distraction rods comprise respectively first and second proximal parts for gripping and pivoting said rods along their respective longitudinal axes L1 and L2.

31. The surgical instrument according to claim 18, wherein it comprises a device for measuring the distance separating the first and second distal parts.

32. The surgical instrument according to claim 31, wherein the measuring device is mounted around the cannula, and wherein said measuring device is disposed between the first intermediate portion and the first proximal part of the first distraction rod, the first distraction rod comprising a portion of its length mounted through said device.

33. The surgical instrument according to claim 31, wherein the measuring device comprises a member for securing the first and second proximal ends together in a determined pivoting position.

34. The surgical instrument according to claim 18, wherein it comprises a securing area configured to cooperate with a securing tool in order to secure said instrument to a support.

35. The surgical instrument according to claim 31, wherein the measuring device comprises a flange comprising graduations.

36. The surgical instrument according to claim 33, wherein the measuring device comprises a rack member for securing the first and second proximal ends together in a determined pivoting position.
